(19) United States
(12) Patent Application Publication
(10) Pub. No.: US 2026/0034162 A1
(43) Pub. Date: Feb. 5, 2026

(54) PHARMACEUTICAL COMPOSITION COMPRISING CYCLODEXTRIN COPOLYMER AND USES THEREOF

(71) Applicant: RENATUS INC., Daejeon (KR)

(72) Inventor: Hee Gon KIM, Daejeon (KR)

(73) Assignee: RENATUS INC., Daejeon (KR)

(21) Appl. No.: 19/276,076

(22) Filed: Jul. 22, 2025

Related U.S. Application Data
(60) Provisional application No. 63/677,511, filed on Jul. 31, 2024.

(51) Int. Cl. A61K 31/724; A61K 9/00; A61P 3/06

(52) U.S. Cl. CPC A61K 31/724; A61K 9/0056; A61P 3/06

(57) ABSTRACT

One or more embodiments relate to a pharmaceutical composition including a cyclodextrin copolymer for prevention or treatment of diseases caused by the harmful or toxic effects of cholesterol and a use thereof. The pharmaceutical composition including a cyclodextrin copolymer composed of gamma-cyclodextrin and beta-cyclodextrin copolymer for prevention or treatment of diseases caused by the harmful or toxic effects of cholesterol according to an embodiment may minimize extraction of plasma membrane cholesterol by including the cyclodextrin copolymer, which does not cause cytotoxicity and hemolytic activity to facilitate metabolism and excretion of cholesterol in cells, and effectively suppresses secretion of IL-1β, MCP-1, and TNF-α cytokines, thereby exhibiting an excellent anti-inflammatory effect.

### CROSS REFERENCE TO RELATED APPLICATIONS

This application is based on and claims priority from U.S. Provisional Application No. 63/677,511, filed on Jul. 31, 2024 in the U.S. Patent and Trademark Office, the disclosures of which are incorporated herein in their entirety by reference.

### TECHNICAL FIELD

One or more embodiments relate to a pharmaceutical composition including a cyclodextrin copolymer for prevention or treatment of diseases caused by the harmful or toxic effects of cholesterol and a use thereof.

### BACKGROUND

Cyclodextrin has a cyclic structure consisting of glucopyranose units linked by α-(1,4) bonds, and includes a-cyclodextrin of six units, β-cyclodextrin of seven units, and γ-cyclodextrin of eight units. Since hydroxyl groups bonded to C2 and C3 of cyclodextrin and a hydroxyl group bonded to C6 extend outward in opposite directions, the outer side of the ring is hydrophilic, and as hydrogen groups of C3 and C5 and ether-like oxygen are positioned inward, the inside of the ring is relatively hydrophobic. Cyclodextrin forms inclusion complexes by the inclusion of hydrophobic molecules in the ring, and due to its property of improving water-solubility of the accommodated hydrophobic molecules, cyclodextrin is applied in various fields, such as cosmetics, foods, textiles, environment and catalysts, drug delivery, and pharmaceuticals.

Although the heights of the ring structures of alpha-, beta-, and gamma-cyclodextrin are the same, the number of glucopyranose units thereof are different from one another, and thus the diameters and volumes inside the rings are different from one another. The inside of the ring of alpha-cyclodextrin having six glucopyranose units is the smallest, and the inside of the ring of gamma-cyclodextrin having eight glucopyranose units is the largest. Due to these differences, alpha-cyclodextrin is known to form complexes with relatively small molecules, and gamma-cyclodextrin with relatively large molecules in the ring.

Among the three types of cyclodextrins, beta-cyclodextrin is known to form complex with cholesterol most effectively. At the same time, beta-cyclodextrin is known to exhibit most cytotoxic and hemolytic properties because of its effects on the plasma membrane. Hearing loss caused by hydroxypropyl-beta-cyclodextrin, which is one of the most widely used derivatives of beta-cyclodextrin and under clinical trials as an active pharmaceutical ingredient, is caused by outer hair cell loss in the cochlea. Beta-cyclodextrin excessively extracts cholesterol from the plasma membrane and causes cell membrane instability and collapse. Since the hair cells are highly sensitive to such plasma membrane disruption, cyclodextrin could lead to cytotoxicity and hearing loss.

Therefore, there is a need to develop a drug capable of facilitating the removal of intracellular and extracellular cholesterol while not destroying or disrupting the plasma membrane via extraction of cholesterol thereof.

### DESCRIPTION OF EMBODIMENTS

### Technical Problem

According to an aspect of an embodiment, a pharmaceutical composition for prevention or treatment of diseases caused by the harmful or toxic effects of cholesterol, including a cyclodextrin copolymer composed of gamma-cyclodextrin (γ-cyclodextrin) and beta-cyclodextrin (β-cyclodextrin), wherein the molar ratio of gamma-cyclodextrin:beta-cyclodextrin of the copolymer is in a range of about 30:70 to about 80:20.

According to another aspect of an embodiment, a food composition and a health functional food composition for prevention or alleviation of diseases caused by the harmful or toxic effects of cholesterol, including a cyclodextrin copolymer composed of gamma-cyclodextrin (γ-cyclodextrin) and beta-cyclodextrin (β-cyclodextrin), wherein the molar ratio of gamma-cyclodextrin:beta-cyclodextrin of the copolymer is in a range of about 30:70 to about 80:20.

According to another aspect of an embodiment, a method of preventing or treating diseases caused by the harmful or toxic effects of cholesterol including administering a a cyclodextrin copolymer composed of gamma-cyclodextrin (γ-cyclodextrin) and beta-cyclodextrin (β-cyclodextrin), to a subject, wherein the molar ratio of gamma-cyclodextrin:beta-cyclodextrin of the copolymer is in a range of about 30:70 to about 80:20.

Other objects and advantages of the present disclosure will be apparent by the appended claims and the following detailed description together with the attached drawings. The contents that are not set forth in the specification can be sufficiently recognized and inferred by a person skilled in the art to which the present disclosure belongs or the art similar thereto, and descriptions thereof will be omitted.

### Solution to Problem

Each of the explanations and exemplary embodiments disclosed herein may be applied to other explanations and exemplary embodiments. That is, all of the combinations of various factors disclosed herein belong to the scope of the present disclosure. Furthermore, the scope of the present disclosure should not be limited by the specific disclosure provided herein below.

According to an aspect of an embodiment, provided is a pharmaceutical composition for prevention or alleviation of diseases caused by the harmful or toxic effects of cholesterol including a cyclodextrin copolymer composed of gamma-cyclodextrin (γ-cyclodextrin, GCD) and beta-cyclodextrin (β-cyclodextrin, BCD), wherein the molar ratio of gamma-cyclodextrin:beta-cyclodextrin of the copolymer is in a range of about 30:70 to about 80:20.

In some embodiments, the copolymer may be cross-linked copolymer, random copolymer, alternating copolymer, block copolymer, and/or graft copolymer.

In some embodiments, the copolymer has a molecular weight in a range of about 2.5 kDa to about 500 kDa.

In some embodiments, An average weight molecular weight (hereinafter, also referred to as “molecular weight”) of the gamma-cyclodextrin and beta-cyclodextrin copolymer may be in a range of, for example, about 2.5 kDa to about 500 kDa, about 4 kDa to about 500 kDa, about 5 kDa to about 500 kDa, about 5.5 kDa to about 500 kDa, about 6 kDa to about 500 kDa, about 6.5 kDa to about 500 kDa, about 7 kDa to about 500 kDa, about 8.5 kDa to about 500 kDa, about 10 kDa to about 500 kDa, 2.5 kDa to about 400 kDa, about 4 kDa to about 400 kDa, about 5 kDa to about 400 kDa, about 5.5 kDa to about 400 kDa, about 6 kDa to about 400 kDa, about 6.5 kDa to about 400 kDa, about 7 kDa to about 400 kDa, about 8.5 kDa to about 400 kDa, about 10 kDa to about 400 kDa, 2.5 kDa to about 300 kDa, about 4 kDa to about 300 kDa, about 5 kDa to about 300 kDa, about 5.5 kDa to about 300 kDa, about 6 kDa to about 300 kDa, about 6.5 kDa to about 300 kDa, about 7 kDa to about 300 kDa, about 8.5 kDa to about 300 kDa, about 10 kDa to about 300 kDa, about 2.5 kDa to about 200 kDa, about 4 kDa to about 200 kDa, about 5 kDa to about 200 kDa, about 5.5 kDa to about 200 kDa, about 6 kDa to about 200 kDa, about 6.5 kDa to about 200 kDa, about 7 kDa to about 200 kDa, about 8.5 kDa to about 200 kDa, about 10 kDa to about 200 kDa, about 2.5 kDa to about 100 kDa, about 4 kDa to about 100 kDa, about 5 kDa to about 100 kDa, about 5.5 kDa to about 100 kDa, about 6 kDa to about 100 kDa, about 6.5 kDa to about 100 kDa, about 7 kDa to about 100 kDa, about 8.5 kDa to about 100 kDa, about 10 kDa to about 100 kDa, 2.5 kDa to about 80 kDa, about 4 kDa to about 80 kDa, about 5 kDa to about 80 kDa, about 5.5 kDa to about 80 kDa, about 6 kDa to about 80 kDa, about 6.5 kDa to about 80 kDa, about 7 kDa to about 80 kDa, about 8.5 kDa to about 80 kDa, about 10 kDa to about 80 kDa, 2.5 kDa to about 60 kDa, about 4 kDa to about 60 kDa, about 5 kDa to about 60 kDa, about 5.5 kDa to about 60 kDa, about 6 kDa to about 60 kDa, about 6.5 kDa to about 60 kDa, about 7 kDa to about 60 kDa, about 8.5 kDa to about 60 kDa, about 10 kDa to about 60 kDa, about 2.5 kDa to about 50 kDa, about 4 kDa to about 50 kDa, about 5 kDa to about 50 kDa, about 5.5 kDa to about 50 kDa, about 6 kDa to about 50 kDa, about 6.5 kDa to about 50 kDa, about 7 kDa to about 50 kDa, about 8.5 kDa to about 50 kDa, about 10 kDa to about 50 kDa, about 2.5 kDa to about 25 kDa, about 4 kDa to about 25 kDa, about 5 kDa to about 25 kDa, about 5.5 kDa to about 25 kDa, about 6 kDa to about 25 kDa, about 6.5 kDa to about 25 kDa, about 7 kDa to about 25 kDa, about 8.5 kDa to about 25 kDa, or about 10 kDa to about 25 kDa.

When the molecular weight of the a cyclodextrin copolymer composed of gamma-cyclodextrin and beta-cyclodextrin is out of these ranges, its effect on solubilizing cholesterol and inducing cholesterol efflux in cells may decrease when used alone or in combination with other drugs.

The cyclodextrin copolymer composed of gamma-cyclodextrin and beta-cyclodextrin copolymer refers to a polymer formed of at least two cyclodextrin monomers that are linked by a covalent bond, and, for example, a bifunctional cross-linker such as epichlorohydrin may be used for the cross-linking of the monomers, but embodiments are not limited thereto.

In certain embodiments, the cyclodextrin copolymer composed of gamma-cyclodextrin and beta-cyclodextrin may be composed of one or more gamma-cyclodextrin monomers and one or more beta-cyclodextrin monomers, and the gamma-cyclodextrin monomers and the beta-cyclodextrin monomers may be cross-linked.

In certain embodiments, the cyclodextrin copolymer is a molecule that comprises at least 2 and at most 200 cyclodextrin monomers. Specifically, the cyclodextrin monomer may include a gamma-cyclodextrin monomer and/or a beta-cyclodextrin monomer. Accordingly, the copolymer may include one or more gamma-cyclodextrin monomers and one or more beta-cyclodextrin monomers.

In some aspects, the number of cyclodextrin monomers constituting the copolymer may be, for example, about 2 to 200, about 3 to 200, about 4 to 200, about 5 to 200, about 6 to 200, about 7 to 200, about 8 to 200, about 9 to 200, about 10 to 200, about 2 to 150, about 3 to 150, about 4 to 150, about 5 to 150, about 6 to 150, about 7 to 150, about 8 to 150, about 9 to 150, about 10 to 150, about 2 to 100, about 3 to 100, about 4 to 100, about 5 to 100, about 6 to 100, about 7 to 100, about 8 to 100, about 9 to 100, about 10 to 100, about 2 to 50, about 3 to 50, about 4 to 50, about 5 to 50, about 6 to 50, about 7 to 50, about 8 to 50, about 9 to 50, about 10 to 50, about 2 to 30, about 3 to 30, about 4 to 30, about 5 to 30, about 6 to 30, about 7 to 30, about 8 to 30, about 9 to 30, about 10 to 30, about 2 to 15, about 3 to 15, about 4 to 15, about 5 to 15, about 6 to 15, about 7 to 15, about 8 to 15, about 9 to 15, or about 10 to 15.

In some aspects, the cyclodextrin copolymer composed of gamma-cyclodextrin and beta-cyclodextrin is a mixture of molecules that comprise at least 2 and at most 200 cyclodextrin monomers. For example, the copolymer may include at least one of a dimer, a trimer, a tetramer, a pentamer, a hexamer, a heptamer, an octamer, a nonamer, and a decamer composed of a beta-cyclodextrin monomer and a gamma-cyclodextrin monomer.

In some aspects, the cyclodextrin copolymer composed of gamma-cyclodextrin and beta-cyclodextrin is a heterodimer. In some aspects, the cyclodextrin copolymer composed of gamma-cyclodextrin and beta-cyclodextrin is a trimer that comprise 2 gamma-cyclodextrin monomers and 1 beta-cyclodextrin monomer, or 2 beta-cyclodextrin monomers and 1 gamma-cyclodextrin monomer. In some aspects, the cyclodextrin copolymer composed of gamma-cyclodextrin and beta-cyclodextrin is a tetramer that comprise 2 gamma-cyclodextrin monomers and 2 beta-cyclodextrin monomers, 3 gamma-cyclodextrin monomers and 1 beta-cyclodextrin monomer, or 1 gamma-cyclodextrin monomer and 3 beta-cyclodextrin monomers.

In some aspects, the cyclodextrin copolymer composed of gamma-cyclodextrin and beta-cyclodextrin comprises only dimers. In some aspects, the cyclodextrin copolymer composed of gamma-cyclodextrin and beta-cyclodextrin comprises only trimers. In some aspects, the cyclodextrin copolymer composed of gamma-cyclodextrin and beta-cyclodextrin comprises only tetramers. In some aspects, the cyclodextrin copolymer composed of gamma-cyclodextrin and beta-cyclodextrin comprises only pentamers. In some aspects, the cyclodextrin copolymer composed of gamma-cyclodextrin and beta-cyclodextrin comprises only hexamers. In some aspects, the cyclodextrin copolymer composed of gamma-cyclodextrin and beta-cyclodextrin comprises only heptamers. In some aspects, the cyclodextrin copolymer composed of gamma-cyclodextrin and beta-cyclodextrin comprises only octamers. In some aspects, the cyclodextrin copolymer composed of gamma-cyclodextrin and beta-cyclodextrin comprises only nonamers. In some aspects, the cyclodextrin copolymer composed of gamma-cyclodextrin and beta-cyclodextrin comprises only decamers.

In some aspects, the gamma-cyclodextrin monomer is gamma-cyclodextrin or its derivatives. In some aspects, the derivative may be induced by replacing at least one hydrogen in an unsubstituted mother group with another atom or a functional group. Specifically, when a functional group is considered to be “substituted”, it means that the functional group may be substituted with at least one substituent selected from halides, C1-C40 alkyl groups, C2-C40 alkenyl groups, C2-C40 alkynyl groups, C3-C40 cycloalkyl groups, C3-C40 cycloalkenyl groups, and C3-C40 aryl groups. For example, the hydrogen can be replaced with C1-C10 linear or branched alkyl, hydroxy C1-C10 linear or branched alkyl, sulfobutylether C1-C10 linear or branched alkyl, or carboxy C1-C10 linear or branched alkyl; particularly, C1-C5 linear or branched alkyl, hydroxy C1-C5 linear or branched alkyl, sulfobutylether C1-C5 linear or branched alkyl, or carboxy C1-C5 linear or branched alkyl; or more particularly, methyl, hydroxypropyl, sulfobutylether, or carboxy methyl, but embodiments are not limited thereto.

In some aspects, the gamma-cyclodextrin monomer may be gamma-cyclodextrin or a derivative thereof represented by the following Formula 1.

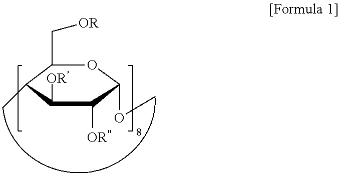

R, R′ and R″ bonded to hydroxyl groups of C2, C3, and C6 of Formula 1 may each independently be, for example, hydrogen, C1-C10 linear or branched alkyl, hydroxy C1-C10 linear or branched alkyl, sulfobutylether C1-C10 linear or branched alkyl, or carboxy C1-C10 linear or branched alkyl; particularly, may be hydrogen, C1-C5 linear or branched alkyl, hydroxy C1-C5 linear or branched alkyl, sulfobutylether C1-C5 linear or branched alkyl, or carboxy C1-C5 linear or branched alkyl; or more particularly, may be hydrogen, methyl, hydroxypropyl (specifically, 2-hydroxypropyl), sulfobutylether, or carboxy methyl, but embodiments are not limited thereto.

When the substituted hydrogen per one glucose is shown as a molar substitution, the molar substitution of the gamma-cyclodextrin derivative may be between 0.2 and 2. In some embodiments, the molar substitution value may be between, for example, about 0.2 to about 2, about 0.3 to about 2, about 0.4 to about 2, about 0.5 to about 2, about 0.6 to about 2, about 0.7 to about 2, about 0.2 to about 1.5, about 0.3 to about 1.5, about 0.4 to about 1.5, about 0.5 to about 1.5, about 0.6 to about 1.5, about 0.2 to about 1.0, about 0.3 to about 1.0, about 0.4 to about 1.0, about 0.5 to about 1.0, about 0.2 to about 0.6, about 0.3 to about 0.6, about 0.4 to about 0.6. In some embodiments, the derivative is hydroxypropyl-gamma-cyclodextrin (more specifically, 2-hydroxypropyl-gamma-cyclodextrin) with a molar substitution value between 0.2 and 2.0.

In some aspects, the gamma-cyclodextrin monomer is hydroxypropyl-gamma-cyclodextrin by Formula 2 having a molar substitution in a range of about 0.5 to about 1.0.

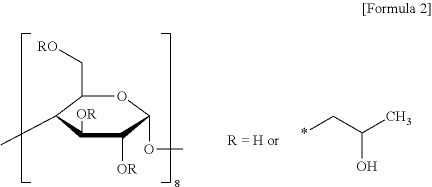

In Formula 2, R bonded to the hydroxyl group of C2, C3, or C6 may each independently be hydrogen or hydroxypropyl (specifically, 2-hydroxypropyl). For example, one, two or three R's bonded to the hydroxyl group of C2, C3 or C6 may be hydroxypropyl (specifically, 2-hydroxypropyl).

In some aspects, the beta-cyclodextrin monomer is beta-cyclodextrin or its derivatives. In some aspects, the derivative may be induced by replacing at least one hydrogen in an unsubstituted mother group with another atom or a functional group. Specifically, when a functional group is considered to be “substituted”, it means that the functional group may be substituted with at least one substituent selected from halides, C1-C40 alkyl groups, C2-C40 alkenyl groups, C2-C40 alkynyl groups, C3-C40 cycloalkyl groups, C3-C40 cycloalkenyl groups, and C3-C40 aryl groups. For example, the hydrogen can be replaced with C1-C10 linear or branched alkyl, hydroxy C1-C10 linear or branched alkyl, sulfobutylether C1-C10 linear or branched alkyl, or carboxy C1-C10 linear or branched alkyl; particularly, C1-C5 linear or branched alkyl, hydroxy C1-C5 linear or branched alkyl, sulfobutylether C1-C5 linear or branched alkyl, or carboxy C1-C5 linear or branched alkyl; or more particularly, methyl, hydroxypropyl, sulfobutylether, or carboxy methyl, but embodiments are not limited thereto.

In some aspects, the beta-cyclodextrin monomer may be beta-cyclodextrin or a derivative thereof represented by the following Formula 3.

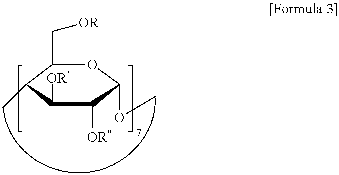

R, R′ and R″ bonded to hydroxyl groups of C2, C3, and C6 of Formula 2 may each independently be, for example, hydrogen, C1-C10 linear or branched alkyl, hydroxy C1-C10 linear or branched alkyl, sulfobutylether C1-C10 linear or branched alkyl, or carboxy C1-C10 linear or branched alkyl; particularly, may be hydrogen, C1-C5 linear or branched alkyl, hydroxy C1-C5 linear or branched alkyl, sulfobutylether C1-C5 linear or branched alkyl, or carboxy C1-C5 linear or branched alkyl; or more particularly, may be hydrogen, methyl, hydroxypropyl (specifically, 2-hydroxypropyl), sulfobutylether, or carboxy methyl, but embodiments are not limited thereto.

In some aspects, the cyclodextrin copolymer composed of gamma-cyclodextrin and beta-cyclodextrinmay may be a copolymer of hydroxypropyl-gamma-cyclodextrin represented by Formula 2 having a molar substitution in a range of about 0.5 to about 1.0 and beta-cyclodextrin.

In some aspects, the cyclodextrin copolymer composed of gamma-cyclodextrin and beta-cyclodextrin may be a copolymer of gamma-cyclodextrin and beta-cyclodextrin or its derivatives. In some aspects, the derivative may be induced by replacing at least one hydrogen in an unsubstituted mother group with another atom or a functional group. Specifically, when a functional group is considered to be “substituted”, it means that the functional group may be substituted with at least one substituent selected from halides, C1-C40 alkyl groups, C2-C40 alkenyl groups, C2-C40 alkynyl groups, C3-C40 cycloalkyl groups, C3-C40 cycloalkenyl groups, and C3-C40 aryl groups. For example, the hydrogen can be replaced with C1-C10 linear or branched alkyl, hydroxy C1-C10 linear or branched alkyl, sulfobutylether C1-C10 linear or branched alkyl, or carboxy C1-C10 linear or branched alkyl; particularly, C1-C5 linear or branched alkyl, hydroxy C1-C5 linear or branched alkyl, sulfobutylether C1-C5 linear or branched alkyl, or carboxy C1-C5 linear or branched alkyl; or more particularly, methyl, hydroxypropyl (specifically, 2-hydroxypropyl), sulfobutylether, or carboxy methyl, but embodiments are not limited thereto.

In one embodiment, at least one of the gamma-cyclodextrin, beta-cyclodextrin and cyclodextrin copolymer may be substituted with hydroxypropyl.

In one embodiment, the cross-linking of gamma-cyclodextrin and beta-cyclodextrin may occur by epichlorohydrin, for example, between alkoxide-induced hydroxyl groups, and an example thereof is shown in the following Reaction Scheme 1.

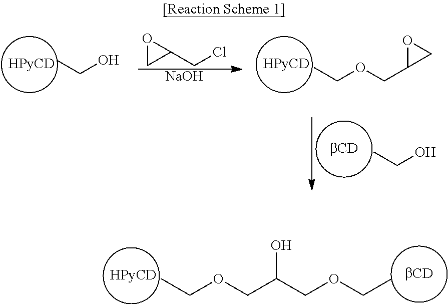

Also, both hydroxypropyl-gamma-cyclodextrin and beta-cyclodextrin may have several alkoxide groups per one monomer, among which random cross-linking occurs, and thus the cross-linked hydroxypropyl-gamma-cyclodextrin and beta-cyclodextrin copolymer may have a linear or cyclic composite structure. Representative examples of the linear-structured copolymer that can be formed are as illustrated in Table 1, but embodiments are not limited thereto. BCD and HPGCD indicate beta-cyclodextrin and hydroxypropyl-gamma-cyclodextrin, respectively.

In some aspects, the molar ratio of gamma-cyclodextrin:beta-cyclodextrin constituting the cyclodextrin copolymer may be in a range of about 5:95 to about 95:5. In some aspects, the molar ratio of hydroxypropyl-gamma-cyclodextrin:beta-cyclodextrin constituting the cyclodextrin copolymer may be in a range of about 5:95 to about 95:5. In some aspects, the molar ratio of hydroxypropyl-gamma-cyclodextrin:beta-cyclodextrin derivative constituting the cyclodextrin copolymer may be in a range of about 5:95 to about 95:5.

In one embodiment, the molar ratio of gamma-cyclodextrin (or hydroxypropyl-gamma-cyclodextrin):beta-cyclodextrin (or beta-cyclodextrin derivative) constituting the cyclodextrin copolymer may be, for example, in a range of 5:95 to 95:5, 5:95 to 90:10, 5:95 to 85:15, 5:95 to 80:20, 5:95 to 75:25, 5:95 to 70:30, 5:95 to 60:40, 5:95 to 50:50, 20:80 to 95:5, 20:80 to 90:10, 20:80 to 85:15, 20:80 to 80:20, 20:80 to 75:25, 20:80 to 70:30, 20:80 to 60:40, 20:80 to 50:50, 25:75 to 95:5, 25:75 to 90:10, 25:75 to 85:15, 25:75 to 80:20, 25:75 to 75:25, 25:75 to 70:30, 25:75 to 60:40, 25:75 to 50:50, 30:70 to 95:5, 30:70 to 90:10, 30:70 to 85:15, 30:70 to 80:20, 30:70 to 75:25, 30:70 to 70:30, 30:70 to 60:40, 30:70 to 50:50, 35:65 to 95:5, 35:65 to 90:10, 35:65 to 85:15, 35:65 to 80:20, 35:65 to 75:25, 35:65 to 70:30, 35:65 to 60:40, 35:65 to 50:50, 40:60 to 95:5, 40:60 to 90:10, 40:60 to 85:15, 40:60 to 80:20, 40:60 to 75:25, 40:60 to 70:30, 40:60 to 60:40, 40:60 to 50:50, 45:55 to 95:5, 45:55 to 90:10, 45:55 to 85:15, 45:55 to 80:20, 45:55 to 75:25, 45:55 to 70:30, 45:55 to 60:40, 45:55 to 50:50, 50:50 to 95:5, 50:50 to 90:10, 50:50 to 80:20, 50:50 to 75:25, 50:50 to 70:30, or 50:50 to 60:40.

In one embodiment, the cyclodextrin copolymer composed of the gamma-cyclodextrin and the beta-cyclodextrin may have cholesterol dissolution activity and cellular cholesterol metabolism and efflux activity, and specifically, may have excellent cholesterol dissolution activity and cellular cholesterol metabolism and efflux activity while having low cytotoxicity and hemolytic activity, and more specifically, may have excellent cholesterol dissolution activity and cellular cholesterol metabolism and efflux activity while having lower cytotoxicity and hemolytic activity than a beta-cyclodextrin (or hydroxypropyl-beta-cyclodextrin) monomer or a polymer thereof.

In one embodiment, the ratio of gamma-cyclodextrin (or hydroxypropyl gamma-cyclodextrin) can be increased to a degree where the hemolytic activity of the synthesized copolymer is lower than the hemolytic activity of the beta-cyclodextrin monomer at the same concentration (mg/ml), but higher than the cholesterol dissolution activity. Specifically, the molar ratio of gamma-cyclodextrin (or hydroxypropyl-gamma-cyclodextrin):beta-cyclodextrin (or beta-cyclodextrin derivative) constituting the cyclodextrin copolymer can be in the range of 5:95 to 80:20.

In one embodiment, the ratio of gamma-cyclodextrin (or hydroxypropyl gamma-cyclodextrin) can be increased to a degree where the hemolytic activity of the synthesized copolymer is lower than the hemolytic activity of the hydroxypropyl-beta-cyclodextrin monomer at the same concentration (mg/ml), but higher than the cholesterol dissolution activity. Specifically, the molar ratio of gamma-cyclodextrin (or hydroxypropyl-gamma-cyclodextrin):beta-cyclodextrin (or beta-cyclodextrin derivative) constituting the cyclodextrin copolymer can be in the range of 30:70 to 95:5.

In one embodiment, the ratio of gamma-cyclodextrin (or hydroxypropyl gamma-cyclodextrin) can be increased to a degree where the hemolytic activity of the synthesized copolymer is lower than the hemolytic activity of the beta-cyclodextrin monomer and/or the hydroxypropyl-beta-cyclodextrin monomer at the same concentration (mg/ml), but higher than the cholesterol dissolution activity. Specifically, the molar ratio of gamma-cyclodextrin (or hydroxypropyl-gamma-cyclodextrin):beta-cyclodextrin (or beta-cyclodextrin derivative) constituting the cyclodextrin copolymer can be in the range of 30:70 to 80:20.

In one embodiment, the hemolytic activity of the cyclodextrin copolymer or a composition comprising the same may be reduced by at least 10% compared to beta-cyclodextrin (BCD), and specifically may be reduced by 10% or more, 20% or more, 30% or more, 50% or more, 70% or more, or 90% or more.

In one embodiment, the cholesterol dissolving activity of the cyclodextrin copolymer or a composition comprising the same may be increased by at least 1.2 times or more than compared to beta-cyclodextrin (BCD), and specifically may be increased by 1.2 times, 1.5 times, 2 times, 5 times or 10 times or more.

In one embodiment, the cross-linking of gamma-cyclodextrin and beta-cyclodextrin may occur by other cross-linking chemistry including click-chemistry and the use of bifunctional cross-linkers.

As used herein, the term “derivative” refers to a compound obtained by substituting a part of a structure of the compound, particularly, a hydroxyl group of C2, C3, or C6, with another atom or atomic group.

As used herein, a derivative may be induced by replacing at least one hydrogen in an unsubstituted mother group with another atom or a functional group. Unless stated otherwise, when a functional group is deemed as “substituted”, it means that the functional group is substituted with at least one substituent selected from halides, C1-C40 alkyl groups, C2-C40 alkenyl groups, C2-C40 alkynyl groups, C3-C40 cycloalkyl groups, C3-C40 cycloalkenyl groups, and C3-C40 aryl groups.

When it is stated as a functional group is “selectively substituted”, it means that the functional group may be substituted with the above substituent.

As used herein, the term “prevention” refers to all actions that suppress diseases caused by the harmful or toxic effects of cholesterol or delay the onset thereof by administration of the pharmaceutical composition according to the present disclosure.

As used herein, the term “treatment” refers to all actions that alleviate or beneficially change symptoms of diseases caused by the harmful or toxic effects of cholesterol by administration of the pharmaceutical composition according to the present disclosure.

As used herein, the term “alleviation” refers to all actions that at least reduce a parameter of diseases caused by the harmful or toxic effects of cholesterol.

As used herein, the term “subject” refers to a target in need of treatment of diseases, and more specifically, a mammal such as a human or a non-human primate, a rodent (e.g., a rat, a guinea pig, etc.), a mouse, a dog, a cat, a horse, a cow, a sheep, a pig, a goat, a camel, or an antelope.

The diseases caused by the harmful or toxic effects of cholesterol may include diseases that occur by abnormal cholesterol metabolism accumulating cholesterol in various tissued in the body such as blood vessels, brain, kidney, liver, lung, eyes, etc. of the body, as normal cholesterol metabolism does not appear, cholesterol dissolution does not appear outside the cell, and intracellular cholesterol metabolism and excretion do not appear. The diseases related to cholesterol metabolism dysregulation may include, for example, those selected from the group consisting of Niemann-pick disease type C, Alzheimer's dementia, Parkinson's disease, focal segmental glomerulosclerosis (FSGS), Alport syndrome, diabetic kidney disease, multiple sclerosis, interstitial pneumonia, osteoarthritis, dyslipidemia, age-related macular degeneration, cardiovascular diseases caused by cholesterol homeostasis dysregulation, metabolic dysfunction-associated steatohepatitis (MASH), and metabolic dysfunction-associated fatty liver disease (MAFLD), and cancer caused by cholesterol homeostasis dysregulation.

The “dyslipidemia” refers to a disease caused by all lipid abnormalities resulting from an increase in low-density lipoprotein cholesterol (LDL-C) and triglycerides (TG) and a decrease in high-density lipoprotein cholesterol (HDL-C). In particular, the dyslipidemia may be at least one selected from the group consisting of hyperlipidemia, hypercholesterolemia, hypertriglyceridemia, combined hyperlipidemia, and combined dyslipidemia, but elements are not limited thereto.

The “hyperlipidemia” refers to a disease caused by an increase in lipids and/or lipoproteins in the blood to an abnormal level. Hyperlipidemia may include hypercholesterolemia, hypertriglyceridemia, and combined hyperlipidemia, according to the type of the increased lipid. The “dyslipidemia” refers to a state of the total cholesterol in the blood has an increase in LDL cholesterol (LDL-C) and triglycerides (TG) and a decrease in HDL cholesterol (HDL-C).

The cardiovascular diseases caused by cholesterol metabolism dysregulation may be, for example, at least one selected from the group consisting of coronary artery disease, peripheral vascular disease, atherosclerotic cardiovascular disease, and cerebrovascular disease, or particularly, for example, at least one selected from the group consisting of hypertension, angina, myocardial infarction, cerebral infarction, stroke, arrhythmia, dyslipidemia, hyperlipidemia, coronary arteriosclerosis, atherosclerosis, and hypercholesterolemia.

The pharmaceutical composition may further include a pharmaceutical additive selected from the group consisting of a diluent, a binder, a disintegrant, a lubricant, and any combination thereof other than the cross-linked gamma-cyclodextrin and beta-cyclodextrin polymer. Also, the pharmaceutical composition may be formulated into injections such as aqueous solutions, suspensions, emulsions, etc., pills, capsules, granules or tablets, with the aid of a diluent, a dispersant, a surfactant, a binder, and/or a lubricant. Also, the pharmaceutical composition may further include a pharmaceutically acceptable carrier, i.e., saline, sterile water, Ringer's solution, buffered saline, cyclodextrin, a dextrose solution, a maltodextrin solution, glycerol, ethanol, liposome, or a mixture of one or more thereof, and if necessary, other common additive such as an antioxidant, a buffer, etc. Moreover, the pharmaceutical composition may be formulated according to respective components using an appropriate method known in the art or a method disclosed in Remington's Pharmaceutical Science (Mack Publishing Company, Easton PA).

The diluent, which may be used to increase quantity, may be selected from the group consisting of mannitol, lactose, starch, microcrystalline cellulose, Ludipress®, calcium dihydrogen phosphate, and any combinations thereof, but embodiments are not limited thereto.

The binder may be selected from the group consisting of povidone, hydroxypropyl cellulose, hydroxypropyl methyl cellulose, polyvinyl alcohol, sodium carboxymethyl cellulose, and any combinations thereof, but embodiments are not limited thereto.

The disintegrant may be selected from the group consisting of croscarmellose sodium, crospovidone, sodium starch glycolate, and any combinations thereof, but embodiments are not limited thereto.

The lubricant may be selected from the group consisting of stearic acid, metal salts of stearic acid (for example, calcium stearate, or magnesium stearate), talc, colloid silica, sucrose fatty acid ester, hydrogenated vegetable oil, wax, glyceryl fatty acid esters, glycerol dibehenate, and any combinations thereof, but embodiments are not limited thereto.

In the pharmaceutical composition, the cyclodextrin copolymer composed of gamma-cyclodextrin and beta-cyclodextrin or a derivative thereof may be included in an amount sufficient to achieve the efficacy or activity. One of ordinary skill in the art may select the upper limit and lower limit of the quantity of the compound contained in the composition within an appropriate range. The pharmaceutical composition may be administered in a dose of, for example, about 0.0001 mg/kg to about 2000 mg/kg, or, to be more effective, about 0.001 mg/kg to about 1000 mg/kg, based on the pharmaceutical composition.

The pharmaceutical composition may be for oral or parenteral administration, and the pharmaceutical composition may be administered orally or parenterally. The pharmaceutical composition may be formulated into an oral or parenteral formulation.

When the pharmaceutical composition is prepared into an oral formulation, the oral formulation may be prepared based on a method for preparing any oral solid dosage as known in the art, specifically, a granule, a pellet, a capsule, or a tablet. The oral formulation may include granules, powders, solutions, tablets, capsules, dry syrups, or combinations thereof. The parenteral formulation may include injectable formulations or external preparations for skin. The external preparations for skin may include creams, gels, ointments, skin emulsions, skin suspensions, transdermal patches, drug-containing bandages, lotions, or combinations thereof. Parenteral administration may include, for example, intravenous injection, subcutaneous injection (or administration), muscular injection, intraperitoneal injection, endothelial administration, local administration, intranasal administration, intrapulmonary administration, intracerebral administration, and rectal administration. For example, the pharmaceutical composition may be administered intravenously or subcutaneously for systemic deliver of the composition, and may be administered intrathecally for central nervous system delivery of the pharmaceutical composition. Also, the composition may be administered by any device which can transport active substances to target cells. A dose of the pharmaceutical composition according to an embodiment may vary depending on the patient's weight, age, sex, state of health and diet, the duration of administration, the mode of administration, excretion rate, or severity of disease, and may be easily determined by those skilled in the art. In addition, for clinical administration, the composition of the present disclosure may be prepared into a suitable formulation using a known technique.

The dose of the composition of the present disclosure may be determined by a person skilled in the art based on the condition of the patient and the severity of the disease. In addition, the composition may be formulated in various dosage forms, including powders, tablets, capsules, liquids, injectable solutions, ointments, and syrup formulations, and may be provided in unit-dosage or multi-dosage containers, for example, sealed ampules or vials.

Also, the pharmaceutical composition according to an embodiment may be administered in combination with another drug sequentially or simultaneously.

The pharmaceutical composition according to an embodiment has less properties of causing cell membrane cholesterol extraction, which lowers cytotoxicity and hemolytic activity, has excellent cholesterol-solubilizing ability, and is capable of improving cholesterol metabolism in cells. Also, it is possible to exhibit anti-inflammatory activity by alleviating the inflammatory response of cells through the regulation of cholesterol metabolism, thereby reducing the secretion of inflammatory cytokines. Also, the cyclodextrin copolymer composed of cross-linked gamma-cyclodextrin and beta-cyclodextrin in the pharmaceutical composition according to an embodiment has characteristics of improving cholesterol metabolism and excretion when injected into the body and not exhibiting ototoxicity even at high concentrations.

According to another aspect of an embodiment, provided is a food composition or a health functional food composition for prevention or alleviation of diseases caused by the harmful or toxic effects of cholesterol, the composition including a cyclodextrin copolymer composed of gamma-cyclodextrin and beta-cyclodextrin. The same contents as described above also apply to the descriptions of the composition.

In the present specification, food refers to natural or processed food which contains at least one or more nutrients, preferably, represents an eatable thing somehow processed, and as an ordinary meaning, it represents foods, food additives, health functional foods, and beverage.

The foods to which the cyclodextrin copolymer composed of cross-linked gamma-cyclodextrin and beta-cyclodextrin according to an embodiment may be added, for example, are various foods, beverages, gums, candies, teas, vitamin mixtures, functional foods, etc. In addition, the foods of the present disclosure include special nutrition foods (for example, milk formulas or infants and kids' foods), eatable processed foods, fish cakes, tofu, starch gel, noodle (for example, ramen or noodle), health supplemental foods, flavoring foods (for example, soy sauce, soy bean paste, red pepper paste, or mixed paste), sauces, cookies (for example, snacks), milk products (for example, fermented oil or cheese), other processed foods, kimchi, salt soaked foods (various kimchi or soy bean-soaked vegetable), beverage (for example, fruit or vegetable beverage, tofu, fermented beverage, or ice cream), natural flavoring sauces (for example, dried ramen soup), vitamin mixtures, alcohol beverage, drinks, and other health supplemental foods, but embodiments are not limited thereto. The foods, beverage, or food additives may be manufactured according to an ordinary method.

In the present specification, the functional food represents a food group value-added to make sure that the function of a corresponding food may be more promoted from its original function with the aid of a physical, biochemical, or biological engineering method or a processed food which is designed to have more of the body control function such as a biological prevention rhythm, a disease prevention and recovery that a food composition has. Preferably, the functional food may refer to a food capable of sufficiently exhibiting the body control function in relation to prevention or alleviation of diseases caused by the harmful or toxic effects of cholesterol. The functional food may include a food supplemental additive which is allowable on the basis of food science and may further include an appropriate carrier, an excipient, and a diluent that are generally used in preparation of functional foods.

A mixing amount of active ingredients may be suitably determined depending on the purpose of use, for example, disease prevention or therapeutic treatment. Generally, when manufacturing foods or beverages, the cyclodextrin copolymer composed of cross-linked gamma-cyclodextrin and beta-cyclodextrin copolymer may be added to food alone or in combination with other food or food component, and the mixing amount of the cyclodextrin copolymer composed of cross-linked gamma-cyclodextrin and beta-cyclodextrin copolymer may be appropriately determined according to a purpose of use thereof (for prevention or alleviation). In food or beverage manufacture, the cyclodextrin copolymer composed of cross-linked gamma-cyclodextrin and beta-cyclodextrin may be added in an amount of about 15 parts by weight or less, about 10 parts by weight or less, about 1 part to about 15 parts by weight, about 1 part to about 10 parts by weight, about 1 part to about 5 parts by weight, or about 0.1 parts to about 15 parts by weight, based on the total weight of the composition. However, when consumed for a long period of time for health and sanitary purposes, the composition may be used in an amount below these ranges. Also, because the active ingredient carries no safety risk, the active ingredient may be used in an amount above these ranges.

The food may be formulated into at least one selected from the group consisting of tablets, pills, powders, granules, fine powders, capsules, and solutions by further including one or more of carriers, excipients, diluents, and additives.

Examples of the carriers, excipients, diluents, and additives may be at least one selected from the group consisting of lactose, dextrose, sucrose, sorbitol, mannitol, erythritol, starch, acacia rubber, calcium phosphate, alginate, gelatin, calcium phosphate, calcium silicate, microcrystalline cellulose, polyvinylpyrrolidone, cellulose, polyvinylpyrrolidone, methylcellulose, water, sugar syrup, methylcellulose, methylhydroxy benzoate, propylhydroxy benzoate, talc, magnesium stearate, and mineral oil.

The “health functional food” defined in the present specification refers to foods produced or processed using raw materials or components that have useful functionality in the human body according to the health functional food law, and the term “functional” means that the food is ingested for the purpose of obtaining a beneficial effect for health use such as controlling nutrients or physiological action for the structure and function of the human body.

The health functional food for preventing or alleviating diseases caused by the harmful or toxic effects of cholesterol according to an embodiment may include about 0.01% to about 95%, preferably, about 1% to about 80%, of the cyclodextrin copolymer composed of cross-linked gamma-cyclodextrin and beta-cyclodextrin, based on the total weight of the composition. Also, the health functional food may be produced or processed in the form of tablets, capsules, powders, granules, solutions, or pills in the purpose of preventing or alleviating diseases caused by the harmful or toxic effects of cholesterol.

According to another aspect of an embodiment, a method of preventing or treating diseases caused by the harmful or toxic effects of cholesterol includes administering a cyclodextrin copolymer composed of gamma-cyclodextrin and beta-cyclodextrin or a composition comprising the same to a subject, wherein the molar ratio of gamma-cyclodextrin:beta-cyclodextrin of the copolymer is in a range of about 30:70 to about 80:20. The same contents as described above also apply to the descriptions of the method.

The cyclodextrin copolymer composed of gamma-cyclodextrin and beta-cyclodextrin of the present disclosure may be used alone or in combination with surgery, radiation therapy, chemotherapy, and methods using biological response modifiers for treatment and prevention of diseases caused by the harmful or toxic effects of cholesterol.

### Advantageous Effects of Disclosure

The pharmaceutical composition including a cyclodextrin copolymer composed of gamma-cyclodextrin and beta-cyclodextrin copolymer for prevention or treatment of diseases caused by the harmful or toxic effects of cholesterol according to an embodiment may minimize extraction of plasma membrane cholesterol by including the cyclodextrin copolymer, which does not cause cytotoxicity and hemolytic activity to facilitate metabolism and excretion of cholesterol in cells, and effectively suppresses secretion of IL-1β, MCP-1, and TNF-α cytokines, thereby exhibiting an excellent anti-inflammatory effect. Also, when administered in combination with other drugs, the gamma-cyclodextrin and beta-cyclodextrin copolymer may exhibit excellent cholesterol excretion and anti-inflammatory effect, as compared to those of other cyclodextrin polymers.

Also, the cyclodextrin copolymer composed of gamma-cyclodextrin and beta-cyclodextrin copolymer may be effectively excreted to the outside of the body through glomerular filtration, and even when injected at high doses, hearing loss and systemic side effects do not occur, and thus the copolymer may be used in prevention or treatment of diseases caused by the harmful or toxic effects of cholesterol such as arteriosclerosis, Alzheimer's dementia, focal segmental glomerulosclerosis, Niemann-Pick's disease type C, metabolic dysfunction-associated steatohepatitis (MASH), and metabolic dysfunction-associated fatty liver disease (MAFLD).

### BRIEF DESCRIPTION OF DRAWINGS

FIG. 1 shows representative gel filtration chromatography of cyclodextrin polymers including (A) BCDP, (B) HPG70B30P, (C) HPG80B20P, and (D) HPGCDP.

FIG. 2 is a schematic illustrating the site of action of cyclodextrins. Cyclodextrins can remove cellular cholesterol mainly by 1) extracting cholesterol from the plasma membrane and 2) promoting cholesterol metabolism and efflux from inside the cells.

FIG. 3 shows the hemolytic activity of cyclodextrins. Data are mean±SD. *P<0.05 and ***P<0.001 compared to HPBCD; ###P<0.001 compared to HPGCD; NS indicates not significant compared to HPGCD; One-way ANOVA analysis and Tukey's multiple comparisons test (n=3).

FIG. 4 shows the CC dissolution efficacy of cyclodextrins. Data are mean±SD. ***P<0.001 compared to HPBCD; One-way ANOVA analysis and Tukey's multiple comparisons test (n=3).

A and B of FIG. 5 shows the CC dissolution efficacy of cyclodextrins at varying concentrations of 10 mg/mL, 5 mg/ml, 2.5 mg/mL, 1.25 mg/mL, 0.625 mg/mL, and 0.3125 mg/mL. Data are mean±SD (n=3).

FIG. 6 shows the amounts of cholesterol that remained in the cells after macrophages that had taken up NBD-CC were treated with cyclodextrins at various concentrations, and the cell viability. Data are mean±SD (n=3).

FIG. 7 shows the amounts of cholesterol that remained in the cells after macrophages that had taken up NBD-CC were treated with cyclodextrins at a non-cytotoxic dose. Data are mean±SD. ***P<0.001 compared to HPBCD, #P<0.05, ###P<0.001 compared to BCDP; One-way ANOVA analysis and Tukey's multiple comparisons test (n=3).

FIG. 8 shows (A) CC dissolution and (B) the amounts of cholesterol that remained in the cells after macrophage that had taken up NBD-CC were treated with HPG70B30P and HPG80B20P with different molecular weights. Data are mean±SD (n=3).

FIG. 9 shows (A) hemolytic activity and (B) CC dissolution of copolymers synthesized using beta-cyclodextrin derivatives including HPBCD and RMBCD. Data are mean±SD (n=3).

FIG. 10 shows (A) representative gel filtration chromatography of GCD-BCD heterodimer and (B) hemolytic activity and (C) CC dissolution of various CDs. Data are mean±SD ***P<0.001 compared to BCD, ###P<0.001 compared to HPβCD; One-way ANOVA analysis and Tukey's multiple comparisons test (n=3).

FIG. 11 is a schematic illustrating the advantages of cross-linked GCD-BCD copolymer. The GCD-BCD copolymer exhibit favorable properties as cholesterol metabolism modulators because it does not induce plasma membrane cholesterol extraction and disruption thereof and has improved cholesterol-solubilizing efficacy.

FIG. 12 shows (A) representative lipids staining images of zebrafish and (B) quantification of liver fat in a metabolic dysfunction associated steatohepatitis (MASH) model. Data are mean±SD ***P<0.001 compared to HFD; One-way ANOVA analysis and Tukey's multiple comparisons test (n=10).

### DETAILED DESCRIPTION

Reference will now be made in detail to embodiments, embodiments of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of at least one of the associated listed items. Expressions such as “at least one of,” when preceding a list of elements, modify the entire list of elements and do not modify the individual elements of the list.

Hereinafter, an aspect of an embodiment will be described in detail according to examples. However, these examples are provided to illustratively describe an aspect of an embodiment, and the scope of an embodiment is not limited to these examples. The examples of an embodiment may provide more complete description of an embodiment to those of ordinary skill in the art.

### Example 1. Synthesis of Cross-Linked Cyclodextrin Copolymer

Hydroxypropyl-gamma-cyclodextrin and beta-cyclodextrin were dissolved in 10 mL of a NaOH aqueous solution (33% w/w) at different ratios as shown in Table 2. For example, HPG10B90P indicates cross-linked hydroxypropyl-gamma-cyclodextrin and beta-cyclodextrin copolymer synthesized with 0.0027 mol (90% of total cyclodextrin in mol) of BCD and 0.0003 mol (10% of total cyclodextrin in mol) of HPGCD. HPG80B20P indicates copolymer synthesized with 0.0006 mol (20% of total cyclodextrin in mol) of BCD and 0.0024 mol (80% of total cyclodextrin in mol) of HPGCD. For the synthesis of beta-cyclodextrin polymer (BCDP) and hydroxypropyl-gamma-cyclodextrin polymer (HPGCDP), 0.003 mol (100% of total cyclodextrin in mol) of beta-cyclodextrin and hydroxypropyl-gamma-cyclodextrin were used respectively. The cyclodextrin solution was sufficiently stirred for 4 hours at room temperature. Cross-linking of the cyclodextrin was induced by the addition of 1.17 ml of epichlorohydrin for a minimum of 1 hour and up to a maximum of 24 hours. To cease the polymerization, excessive acetone was added thereto, and the resultants were remained at room temperature for 30 minutes. After removing acetone, the resultants were remained in an oven at 50° C. for 16 hours. 2 N HCl was used to adjust the pH of the solution to about 11. Dialysis and ultrafiltration were performed to remove salt and cyclodextrin monomers. The solution was filtered through a 0.22 μm PES filter and then freeze-dried to obtain a powder and stored at room temperature.

TABLE 2

| | BCDP | HPG10B90P | HPG20B80P | HPG30B70P | HPG50B50P | HPG70B30P | HPG80B20P | HPG90B10P | HPGCDP |
|---|---|---|---|---|---|---|---|---|---|
| BCD (mol) | 0.003 | 0.0027 | 0.0024 | 0.0021 | 0.0015 | 0.0009 | 0.0006 | 0.0003 | 0 |
| HPGCD (mol) | 0 | 0.0003 | 0.0006 | 0.0009 | 0.0015 | 0.0021 | 0.0024 | 0.0027 | 0.003 |

### Example 2. Assessment of the Solubility and Molecular Weight of the Cross-Linked Cyclodextrin Copolymer

Since excessive cross-linking and polymerization leads to gelation and production of insoluble polymer, the solubility of the polymers was assessed. The polymers in the powder form were resuspended in the distilled water at various concentrations from 10 mg/mL to 200 mg/mL. As shown in Table 3, whereas beta-cyclodextrin monomer (BCD) had limited solubility in the water, which was less than about 20 mg/mL, the beta-cyclodextrin polymer (BCDP) and hydroxypropyl-gamma-cyclodextrin and beta-cyclodextrin copolymer (HPG10B90P, HPG20B80P, HPG30B70P, HPG50B50P, HPG70B30P, HPG80B20P, HPG90B10P) were greatly soluble in the water (>200 mg/mL). Hydroxypropyl-gamma-cyclodextrin monomer (HPGCD) and its polymer (HPGCDP) were soluble as well.

TABLE 3

| mg/ml | BCD | BCDP | HPG10B90P | HPG20B80P | HPG30B70P | HPG50B50P | HPG70B30P | HPG80B20P | HPG90B10P | HPGCD | HPGCDP |
|---|---|---|---|---|---|---|---|---|---|---|---|
| 10 | ◯ | ◯ | ◯ | ◯ | ◯ | ◯ | ◯ | ◯ | ◯ | ◯ | ◯ |
| 50 | X | ◯ | ◯ | ◯ | ◯ | ◯ | ◯ | ◯ | ◯ | ◯ | ◯ |
| 100 | X | ◯ | ◯ | ◯ | ◯ | ◯ | ◯ | ◯ | ◯ | ◯ | ◯ |
| 200 | X | ◯ | ◯ | ◯ | ◯ | ◯ | ◯ | ◯ | ◯ | ◯ | ◯ |

◯: soluble,
X: insoluble

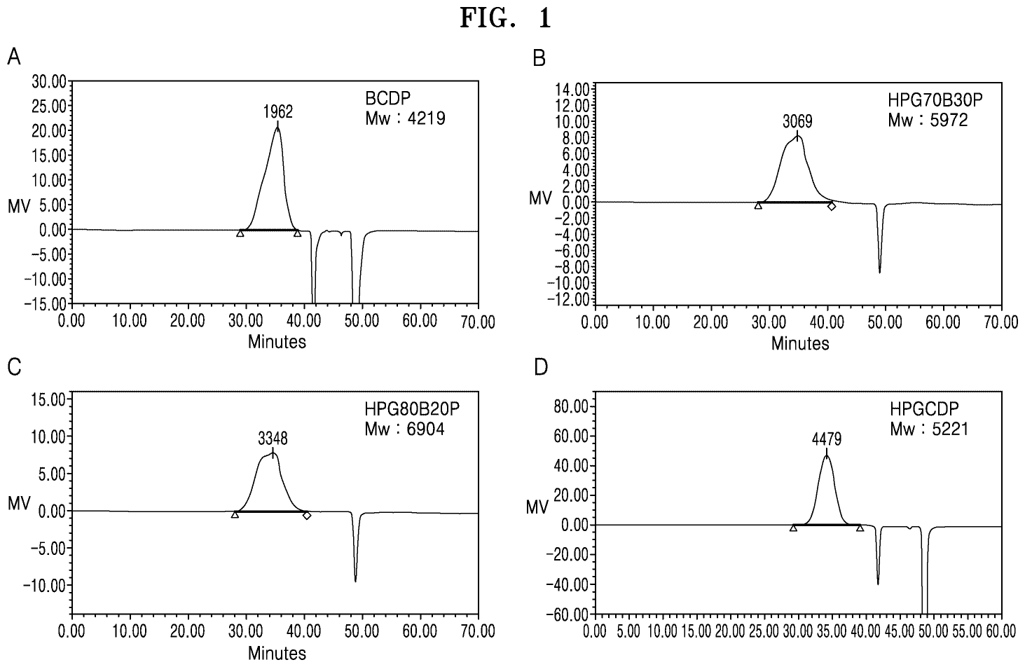

Furthermore, gel filtration chromatography revealed successful cross-linking of cyclodextrins as shown in FIG. 1. Whereas beta-cyclodextrin and hydroxypropyl-gamma-cyclodextrin monomers have a molecular weight of 1135 Da and 1540 Da, respectively, the polymers exhibited increased molecular weight. For example, BCDP, HPG70B30P, HPG80B20P, HPGCDP exhibited a molecular weight of 4219 Da, 5972 Da, 6904 Da, and 5221 Da, respectively.

### Example 3. Assessment of Plasma Membrane Disruption

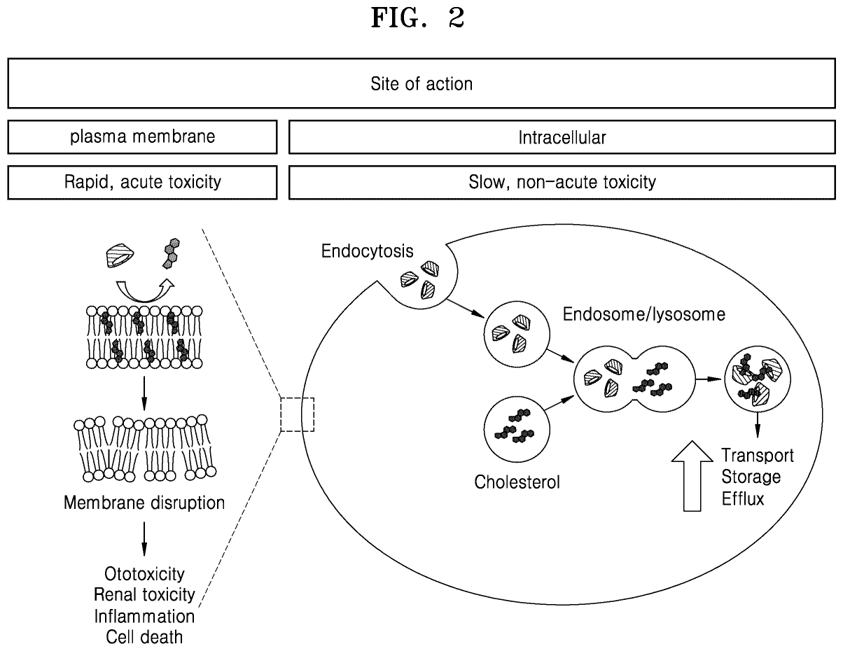

Cyclodextrins can function on the plasma membrane and inside the cells to remove cellular cholesterol. As shown in FIG. 2, depletion of cholesterol from the plasma membrane is rapid and induces acute cytotoxicity whereas promotion of cholesterol metabolism inside the cells is rather slow and less cytotoxic. The dose-limiting toxicity of hydroxypropyl-beta-cyclodextrin (HPBCD), which is under clinical trials for various diseases, is ototoxicity. It is known that loss of outer hair cells in the cochlea due to plasma membrane disruption is the main cause of the ototoxicity and hearing loss. Therefore, strategies to minimize plasma membrane disruption and promote intracellular function of cyclodextrins are in great need.

Hemolysis occurs when the plasma membrane is disrupted and is one of the side effects of cyclodextrins. Among alpha-, beta-, and gamma-cyclodextrin, beta-cyclodextrin is known as the most hemolytic one. However, since it has the highest affinity to cholesterol at the same time, engineering of beta-cyclodextrins has potential to optimize its non-plasma membrane cholesterol removal.

For hemolytic activity analysis, 10 μL of whole blood was mixed with 300 μL of a PBS solution containing 50 mg/ml of cyclodextrin and reacted at 37° C. for 30 minutes. For monomers, HPBCD, BCD, and HPGCD were used. For BCD, 12.5 mg/mL of solution was used due to its limited solubility. After centrifuging the mixture at 3,000 g for 5 minutes, the absorbance at 570 nm was measured using the supernatant. As a positive control, distilled water was used.

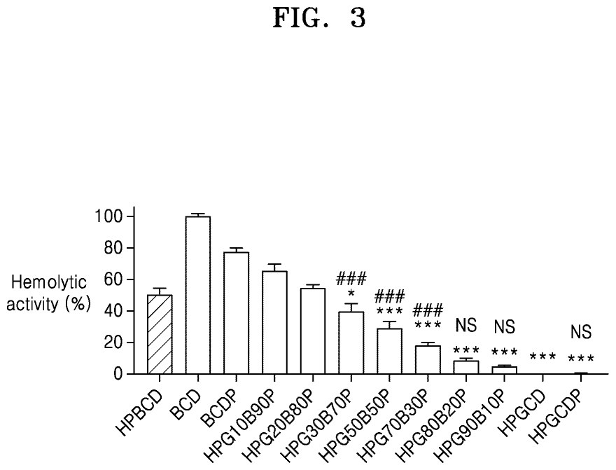

As shown in FIG. 3, the hemolytic activity for monomers HPBCD, BCD, and HPGCD were 50%, 99.7%, and 0%, respectively. For polymers, the hemolytic activity decreased in the order of BCDP, HPG10B90P, HPG20B80P, HPG30B70P, HPG50B50P, HPG70B30P, HPG80B20P, HPG90B10P, and HPGCDP. HPG70B30P, HPG80B20P, HPG90B10P, and HPGCDP exhibited significantly lower hemolytic activity compared to HPBCD. HPG30B70P, HPG50B50P, HPG70B30P exhibited significantly higher hemolytic activity compared to HPGCD whereas HPG80B20P, HPG90B10P showed no significant difference.

### Example 4. Assessment of Cholesterol Crystal (CC) Dissolution

To investigate whether the reduced hemolytic activity and extraction of cholesterol from the plasma membrane should mean reduced ability to interact with non-plasma membrane cholesterol, CC dissolution assays were performed.

CC was prepared by crystallization of powdered cholesterol from ethanol. 10 mg of cholesterol powder was dissolved in 5 mL ethanol. 7.5 mL distilled water was added to induce crystallization and the mixture was freeze-dried. The CC was resuspended in the aqueous buffer with sonication and vortexing.

For CC dissolution analysis, 10 μg of CC was mixed with 300 μL of a PBS solution containing 10 mg/ml of cyclodextrin and reacted at 37° C. for 30 minutes. For monomers, HPBCD, BCD, and HPGCD were used. The solution was filtered through a 0.22 μm PES syringe filter and the filtrate was used for the quantification of dissolved cholesterol using Amplex Red Cholesterol Assay Kit by measuring the fluorescence at 540 nm/590 nm.

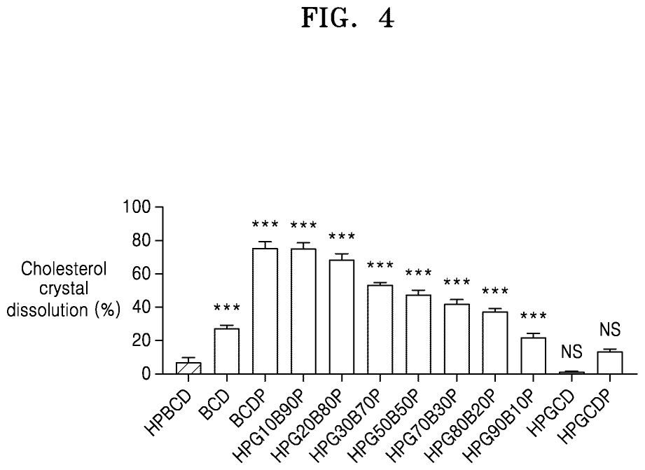

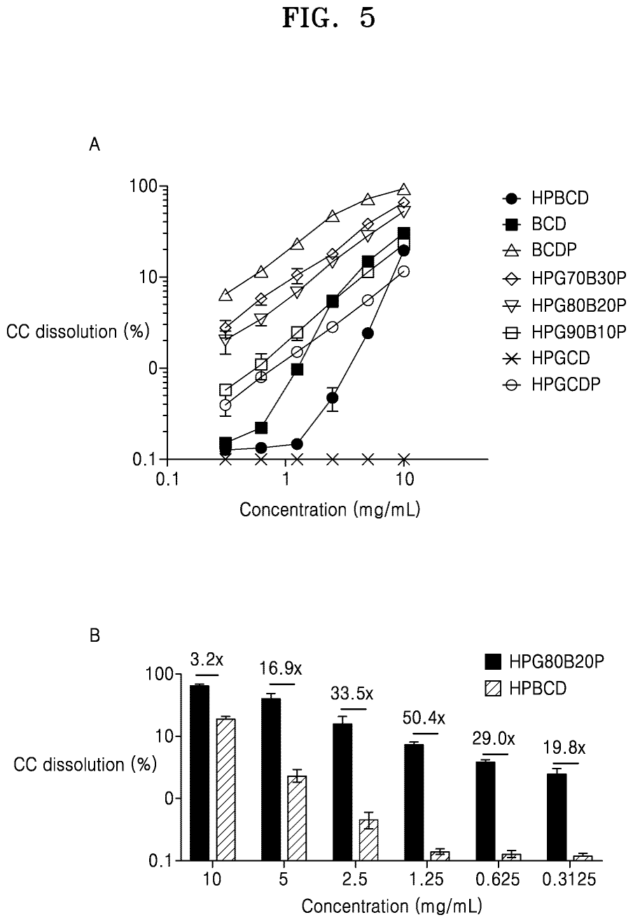

As shown in FIG. 4, the CC dissolution for monomers HPBCD, BCD, and HPGCD were 6.7%, 27.3%, and 1.3%, respectively. For polymers, the CC dissolution efficacy decreased in the order of BCDP, HPG10B90P, HPG20B80P, HPG30B70P, HPG50B50P, HPG70B30P, HPG80B20P, HPG90B10P, and HPGCDP. Polymers including BCDP, HPG10B90P, HPG20B80P, HPG30B70P, HPG50B50P, HPG70B30P, HPG80B20P, and HPG90B10P exhibited significantly higher CC dissolution efficacy compared to HPBCD.

Furthermore, as shown in FIG. 5A, whereas the monomers including HPBCD, BCD, and HPGCD showed exponential decrease in the efficacy in cholesterol dissolution as the concentration decreases, polymers including BCDP, HPG70B30P, HPG80B20P, HPG90B10P, and HPGCDP showed linear decrease in the efficacy. As shown in FIG. 5B, when the CC dissolution efficacy of HPBCD and HPG80B20P at varying concentrations were compared, HPG80B20P exhibited at least 3.2-fold to at most 50.4-fold higher efficacy.

The exponential decrease in the efficacy of the monomers in cholesterol dissolution can be explained by the fact that cyclodextrin monomers tend to interact with cholesterol at a 1:1 (cyclodextrin:cholesterol) molar ratio at low concentrations while they tend to interact at a 2:1 molar ratio, which provides more stable solubilization of cholesterol by capping both hydrophobic ends of cholesterol, at high concentrations. The linear decrease in the efficacy of polymers in cholesterol dissolution can be explained by the fact that the covalent cross-linking of cyclodextrins allow the formation of 2:1 inclusion complex at low concentrations.

Overall, when combining both results of hemolytic activity and CC dissolution, cross-linked HPGCD and BCD including HPG70B30P, HPG80B20P, HPG90B10P exhibited significantly lower hemolytic activity and significantly higher CC dissolution efficacy compared to HPBCD.

### Example 5. Assessment of Cellular Cholesterol Efflux and Viability

CC containing NBD-cholesterol (NBD-CC) was prepared by crystallization of powdered cholesterol and NBD-cholesterol from ethanol. 9 mg of cholesterol and 1 mg of NBD-cholesterol powder was dissolved in 5 mL ethanol. 7.5 ml distilled water was added to induce crystallization and the mixture was freeze-dried. The NBD-CC was resuspended in the aqueous buffer with sonication and vortexing.

Raw 264.7 cells were dispensed at 5×104 cells per well of a 96-well plate, and after 24 hours, the cells were treated with cholesterol crystals at a concentration of 50 μM. After 3 hours, the cholesterol crystals not ingested into the cells were removed through medium replacement and then treated with cyclodextrin at varying concentrations. The cells were cultured at 37° C. in 5% CO₂ for 48 hours, and intracellular cholesterol fluorescence was analyzed after lysing the cells and measuring the fluorescence of the lysate at 480 nm/530 nm. PBS-treated cells were used as a negative control.

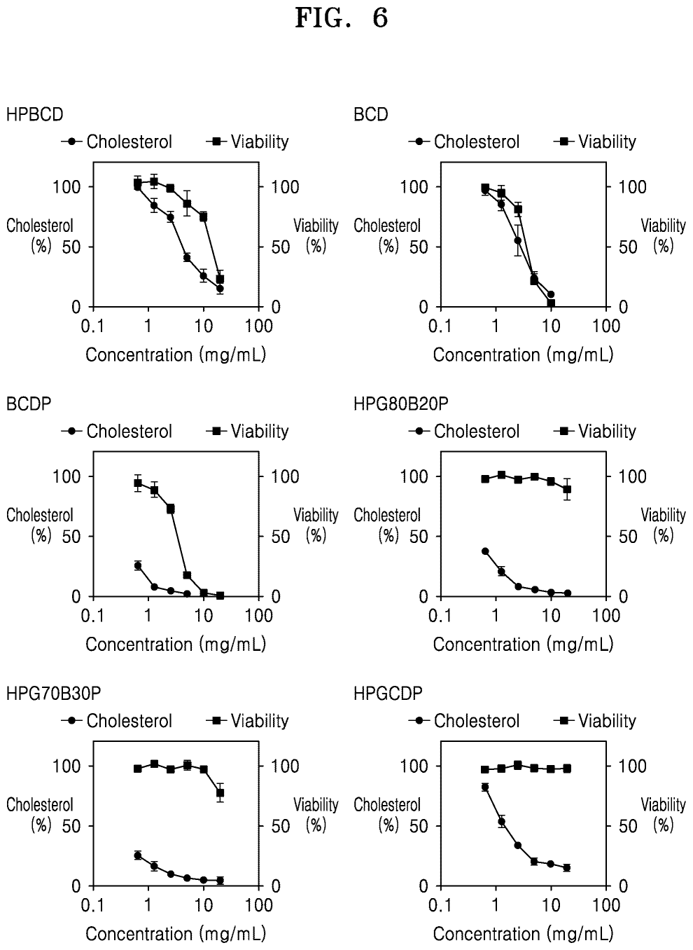

As shown in FIG. 6, HPBCD, BCD, and BCDP showed dose-dependent cytotoxicity and resulted in almost complete cell loss at the highest concentration of 20 mg/mL. HPG80B20P, HPG70B30P, and HPGCDP, which exhibited low hemolytic activity showed no significant cytotoxicity. At all concentrations, copolymers HPG80B20P and HPG70B30P showed higher cholesterol efflux efficiency compared to HPGCDP. At the lowest concentration of 0.625 mg/mL, HPG80B20P, HPG70B30P, and HPGCDP showed remaining cholesterol of 37.6%, 24.9%, and 82.3%, respectively.

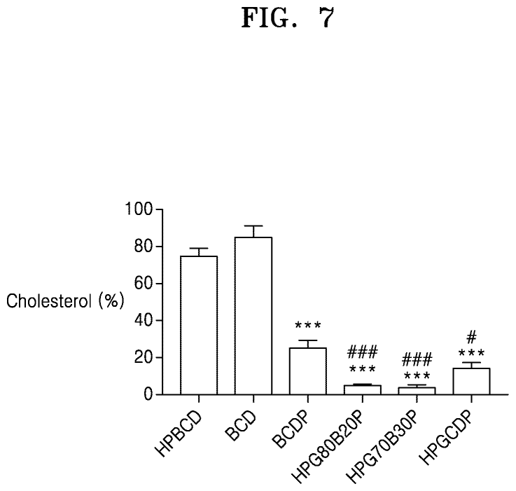

As shown in FIG. 7, at non-cytotoxic doses, which were 1.25 mg/mL for HPBCD, 0.625 mg/mL for BCD, 0.625 mg/mL for BCDP, 10 mg/mL for HPG80B20P, 10 mg/mL for HPG70B30P, and 20 mg/mL for HPGCDP, HPG80B20P and HPG70B30P showed most significant NBD-CC reduction in the cells. The results show that cross-linked HPGCD and BCD copolymer including HPG70B30P and HPG80B20P can induce effective cholesterol efflux without significant cyclodextrin-induced cytotoxicity.

Overall, these results suggest that the strategy to cross-link different cyclodextrin monomer units, beta-cyclodextrin with an excellent affinity to cholesterol and hydroxypropyl-gamma-cyclodextrin with almost no cytotoxicity, at an optimal molar ratio led to the discovery of novel cyclodextrins with great potential to modulate cellular cholesterol metabolism and with much less toxicity compared to conventionally available cyclodextrins.

### Example 6. Assessment of the Impact of Average Molecular Weight of the Copolymer on CC Dissolution and Cholesterol Efflux

The average molecular weight of HPG70B30P and HPG80B20P mainly used for the experiments was 5.9 kDa and 6.9 kDa, respectively, as determine by gel filtration chromatography.

To assess the impact of average molecular weights of the copolymer on CC dissolution and cholesterol efflux, synthesis of copolymers of different molecular weights were performed by the addition of different amounts of epichlorohydrin. The molar ratios of HPGCD to BCD used in the experiment were the ones used for the synthesis of HPG70B30P and HPG80B20P. After dissolving HPGCD and BCD in 10 mL of distilled water, the cyclodextrin solution was sufficiently stirred for 4 hours at room temperature. Cross-linking of the cyclodextrin was induced by the addition of 0.47 mL, 0.70 mL, 1.17 mL, 1.64 mL, 2.12 mL, 2.81 mL, and 3.51 mL of epichlorohydrin for at least 1 hour and up to 24 hours. To cease the polymerization, excessive acetone was added thereto, and the resultants were remained at room temperature for 30 minutes. After removing acetone, the resultants were remained in an oven at 50° C. for 16 hours. 2 N HCl was used to adjust the pH of the solution to about 11. Dialysis and ultrafiltration were performed to remove salt and cyclodextrin monomers. The solution was filtered through a 0.22 μm PES filter and then freeze-dried to obtain a powder and stored at room temperature. The average molecular weights of HPG70B30P were 5.9 kDa, 13.2 kDa, 40.9 kDa, 125.3 kDa, and 302.3 kDa. The average molecular weights of HPG80B209 were 6.9 kDa, 15.6 kDa, 45.9 kDa, 137 kDa, and 352.3 kDa.

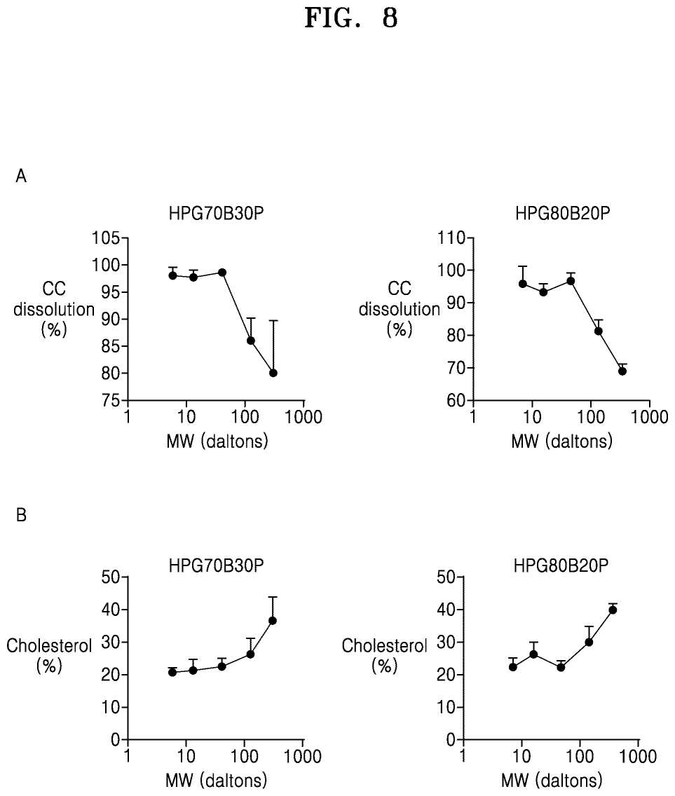

As shown in FIG. 8, the efficacy of HPG70B30P in CC dissolution was in the order of 5.9 kDa, 40.9 kDa, 13.2 kDa, 125.3 kDa, and 352.3 kDa. The efficacy of HPG80B20P in CC dissolution was in the order of 45.9 kDa, 6.9 kDa, 15.6 kDa, 137 kDa, and 352.3 kDa. The efficacy of HPG70B30P in cholesterol efflux was in the order of 5.9 kDa, 13.2 kDa, 40.9 kDa, 125.3 kDa, and 352.3 kDa. The efficacy of HPG80B20P in cholesterol efflux was in the order of 6.9 kDa, 45.9 kDa, 15.6 kDa, 45.9 kDa, and 137 kDa. The results suggest that the average molecular weight of the copolymer can be optimized by manipulating the degree of cross-linking to have optimal CC dissolution and cholesterol efflux efficiency.

### Example 7. CC Dissolution and Hemolytic Activity of Various Copolymers

We prepared various copolymers composed of 1) GCD and BCD, 2) HPGCD and hydroxypropyl-beta-cyclodextrin (HPBCD), 3) HPGCD and methylated-beta-cyclodextrin (RMBCD). The molar ratio of GCD to BCD was 2:1. Briefly, 0.0024 mol of GCDs (e.g., GCD and HPGCD) and 0.0012 mol of BCDs (e.g., BCD, HPBCD, RMBCD) were dissolved in 10 mL of 25 to 33% NaOH solution and stirred for 4 hours. Then epichlorohydrin was added and the mixture was incubated for 20 hours. The molar ratio of epichlorohydrin to CD ranged from 5 (HPGCD-HPBCD and HPGCD, RMBCD copolymers) to 10 (GCD-BCD copolymer). The rest of the process for the synthesis of the copolymer was the same as described in Example 1.

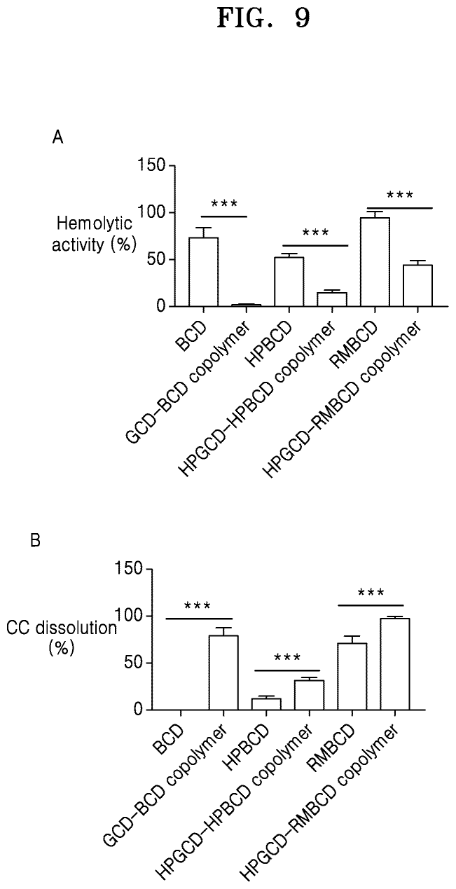

As shown in FIG. 9, GCD-BCD, HPGCD-HPBCD, and HPGCD-RMBCD copolymer showed significantly reduced hemolytic activity and increased CC dissolution efficacy compared to HPBCD and RMBCD monomer, respectively. The concentrations of cyclodextrin were 50 mg/mL (15 mg/mL for BCD due to its low solubility), and 10 mg/mL for hemolytic activity and CC dissolution tests, respectively. Although BCD monomers had low solubility and therefore almost no capacity in solubilizing cholesterol, GCD-BCD copolymer exhibited excellent CC dissolution capacity.

Lastly, we to investigate whether the enhanced CC dissolution capacity and reduced hemolytic activity can be achieved using other cross-linkers, we formed GCD and BCD heterodimer using copper-catalyzed azide-alkyne cycloadditon (CuAAC), or click-chemistry. To prepare the BCD-GCD heterodimer, we began with the functionalization of BCD with an azide (—N₃) group and GCD with an alkyne (—C≡C) group. First, BCD was dissolved in anhydrous DMF and sodium hydride (NaH) was added carefully to deprotonate the hydroxyl groups selectively at the O2 position, making them more reactive. Once deprotonation was complete, tosyl chloride (TsCl) was added to introduce a tosyl leaving group, forming a tosylated BCD intermediate. The TsCl reaction was performed at 0° C. for 4 hours. This intermediate was then reacted with sodium azide (NaN₃) at 60° C. in DMF for 12 hours, allowing the nucleophilic substitution of the tosyl group by an azide (—N₃). The product was purified by dialysis membrane with a 3 kDa molecular weight cut-off, effectively removing excess reactants and unwanted side products. Second, GCD was dissolved in DMF, and NaH was added to selectively deprotonate the hydroxyl groups, enabling regioselective substitution. Following this, propargyl bromide to GCD was introduced to incorporate an alkyne (—C≡C) functionality via nucleophilic substitution. The reaction was stirred at room temperature to 50° C. for 12 hours to ensure full conversion. Once complete, the alkyne-functionalized GCD was purified through dialysis membrane with a 3 kDa molecular weight cut-off. Then, the BCD-N₃ and GCD-C≡C were dissolved in a water/t-butanol (1:1) solvent mixture, which provides an optimal environment for the Cu(I)-catalyzed azide-alkyne cycloaddition (CuAAC) reaction. A Cu(I) catalyst is introduced—this can be generated in situ using CuSO₄ and sodium ascorbate, or CuBr with TBTA ligand can be directly added for improved efficiency. The reaction was allowed to proceed under gentle stirring at room temperature or 40° C. for 12-24 hours to enable the selective formation of a triazole linkage between BCD and GCD. After the reaction is complete, excess copper was removed using EDTA treatment. The BCD-GCD heterodimer was further purified via dialysis membrane with a 5 kDa molecular weight cut-off.

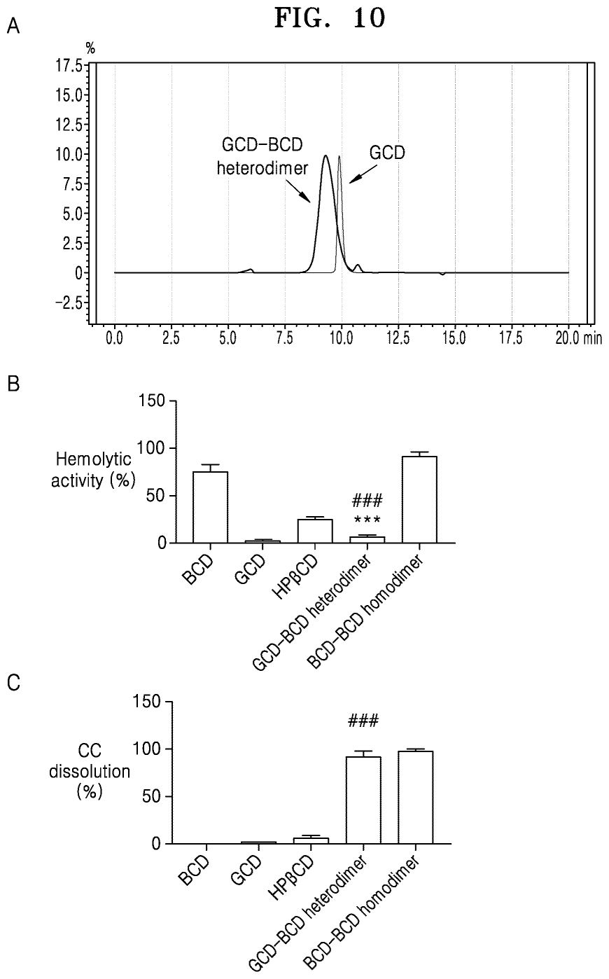

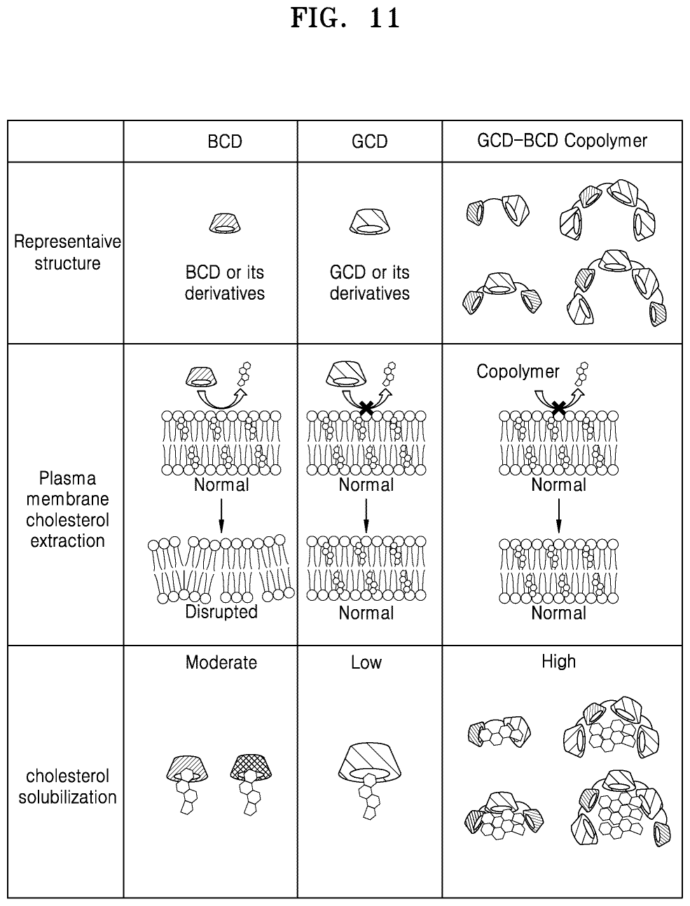

As shown in FIG. 10, gel filtration chromatography revealed successful formation of GCD-BCD heterodimer, and the average molecular weight is approximately 2.5 kDa. Hemolytic activity of GCD-BCD heterodimer was significantly lower than BCD and HPβCD monomers. On the other hand, BCD-BCD homodimer, which was synthesized with the click chemistry used for the synthesis of GCD-BCD heterodimer, induced very significant hemolytic activity. The CC dissolution efficacy of GCD-BCD copolymer was superior to the monomers. Overall, these results show that cross-linking of BCD or BCD derivatives that exhibit high affinity for cholesterol and high cytotoxicity with GCD or GCD derivatives that exhibit moderate affinity for cholesterol and almost no cytotoxicity can lead to generation of a novel cyclodextrin copolymer with a molecular structure allowing minimal plasma membrane cholesterol extraction and maximal cholesterol solubilization as shown in FIG. 11. In other words, these data suggest that the cross-linked gamma-cyclodextrin and beta-cyclodextrin copolymer can provide a platform technology to reduce cytotoxicity of cyclodextrin while improving cholesterol-solubilizing capacities. The technology will facilitate the application of cyclodextrins produced thereby for prevention or treatment of cholesterol metabolic diseases, offering a wider therapeutic window compared to conventional cyclodextrins.

### Example 8. Evaluation of Therapeutic Efficacy In Vivo

To investigate the effect of various CDs, metabolic dysfunction-associated fatty liver disease (MALFD) was induced in Zebrafish with high-fat diet (HFD). While the normal fat diet (NFD) included Gemma micro ZF 75, the HFD included 40% egg yolk powder, which is very abundant with cholesterol and other lipids. From 5 days post-fertilization (dpf) to 22 dpf, the diet was provided three times a day at a dosage of 1 mg per 10 fish per feeding. Drug was administered from 15 dpf to 22 dpf. Resmetirom was used as a positive control. The doses for resmetirom and CD were 3 μg/mL and 2 mg/mL, respectively. The GCD-BCD copolymer had an average molecular weight of approximately 5 kDa. Zebrafish exposed to the drug were transferred to fresh medium at 22 days post-fertilization (dpf) and fasted for 24 hours. They were then fixed overnight in 4% paraformaldehyde (PFA), washed with PBS, and stained with 0.5% Oil Red O at room temperature, followed by washing with isopropyl alcohol. Stained zebrafish were mounted by gradually increasing the glycerol concentration from 20% to 100% and imaged under a stereo microscope under identical lighting conditions. To quantify the Oil Red O staining intensity in the liver region of zebrafish larvae, the grey mean value was measured using ImageJ.

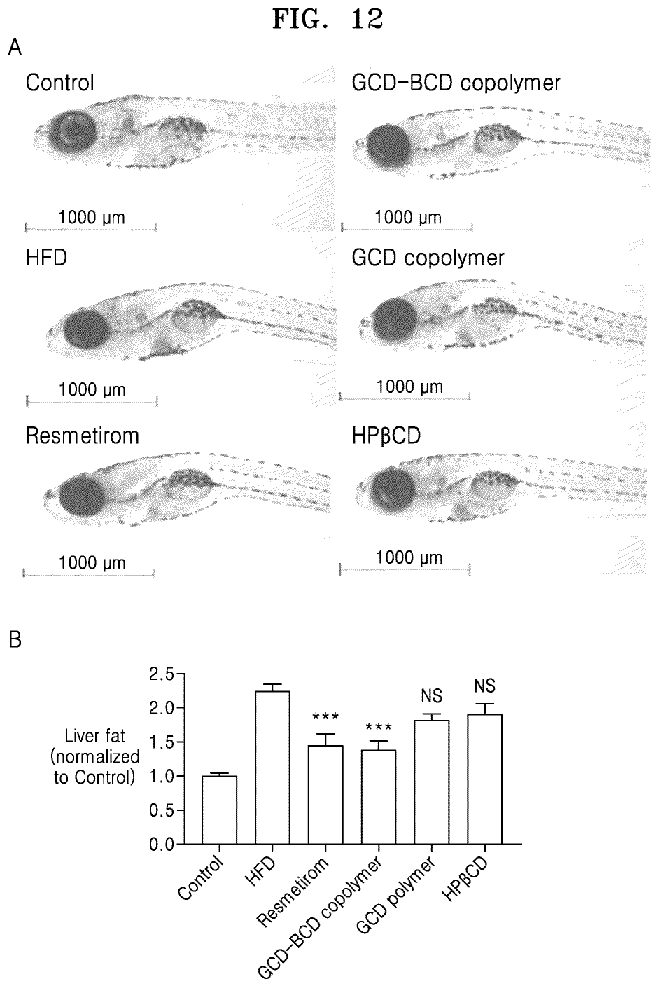

As shown in FIG. 12, zebrafish developed significant liver fat under a HFD. In fish treated with either resmetirom or GCD-BCD copolymer, there was a significant decrease in liver fat, whereas GCD polymer or HPβCD showed no significant effect. These results indicate that GCD-BCD copolymer is effective in reducing liver fat.

What is claimed is:

1. A pharmaceutical composition for prevention or treatment of diseases caused by the harmful or toxic effects of cholesterol, comprising a cyclodextrin copolymer composed of gamma-cyclodextrin (γ-cyclodextrin) and beta-cyclodextrin (β-cyclodextrin),
wherein the molar ratio of gamma-cyclodextrin:beta-cyclodextrin of the copolymer is in a range of about 30:70 to about 80:20.

2. The pharmaceutical composition of claim 1, wherein the copolymer has a molecular weight in a range of about 2.5 kDa to about 500 kDa.

3. The pharmaceutical composition of claim 1, wherein the copolymer is cross-linked copolymer, random copolymer, alternating copolymer, block copolymer, and/or graft copolymer.

4. The pharmaceutical composition of claim 1, wherein the copolymer comprises at least 2 to at most 200 cyclodextrin monomers.

5. The pharmaceutical composition of claim 1, wherein the γ-cyclodextrin, β-cyclodextrin, and/or the copolymer is substituted with hydroxypropyl.

6. The pharmaceutical composition of claim 1, wherein the copolymer is a heterodimer of γ-cyclodextrin and β-cyclodextrin.

7. The pharmaceutical composition of claim 1, wherein the diseases caused by the harmful or toxic effects of cholesterol are selected from the group consisting of Niemann-pick disease type C, Alzheimer's dementia, Parkinson's disease, focal segmental glomerulosclerosis (FSGS), Alport syndrome, diabetic kidney disease, multiple sclerosis, interstitial pneumonia, osteoarthritis, dyslipidemia, cardiovascular diseases caused by dysregulated cholesterol homeostasis, metabolic dysfunction-associated steatohepatitis (MASH), metabolic dysfunction-associated fatty liver disease (MAFLD), and cancer caused by dysregulated cholesterol homeostasis.

8. The pharmaceutical composition of claim 1, wherein the hemolytic activity of the composition is reduced by at least 10% compared to beta-cyclodextrin (BCD).

9. The pharmaceutical composition of claim 1, wherein the cholesterol dissolution activity of the composition is increased by at least 1.2 times compared to beta-cyclodextrin (BCD).

10. A food composition for prevention or alleviation of diseases caused by the harmful or toxic effects of cholesterol, comprising a cyclodextrin copolymer composed of γ-cyclodextrin and β-cyclodextrin,
wherein the molar ratio of gamma-cyclodextrin:beta-cyclodextrin of the copolymer is in a range of about 30:70 to about 80:20.

11. A method of treating, preventing or alleviating diseases caused by the harmful or toxic effects of cholesterol, the method comprising administering a cyclodextrin copolymer composed of γ-cyclodextrin and β-cyclodextrin copolymer to a subject in need thereof,
wherein the molar ratio of gamma-cyclodextrin:beta-cyclodextrin of the copolymer is in a range of about 30:70 to about 80:20.
